(19) United States
(12) Patent Application Publication
(10) Pub. No.: US 2015/0126837 A1
(43) Pub. Date: May 7, 2015

(54) DEVICES, SYSTEMS, AND METHODS TO DETERMINE VOLUME REFLUX

(71) Applicants: Ghassan S. Kassab, La Jolla, CA (US); Hans Gregersen, Egaa (DK)

(72) Inventors: Ghassan S. Kassab, La Jolla, CA (US); Hans Gregersen, Egaa (DK)

(21) Appl. No.: 14/530,868

(22) Filed: Nov. 3, 2014

Related U.S. Application Data
(60) Provisional application No. 61/898,596, filed on Nov. 1, 2013.

(51) Int. Cl. A61B 5/00; A61B 5/053

(52) U.S. Cl. CPC A61B 5/4233; A61B 5/687; A61B 5/0538; A61B 5/4205; A61B 5/4211
USPC 600/350; 600/380

(57) ABSTRACT

Devices, systems, and methods to determine volume reflux. In an exemplary embodiment of a device of the present disclosure, the device comprises an elongated body having a sufficient length so that a distal end of the elongated body can extend to a gastroesophageal junction of a person while a proximal end of the device, or a connector coupled thereto, is present at or distal to a throat of the person relative to the gastroesophageal junction, and a plurality of least ten electrodes positioned at known distances from one another along the elongated body, wherein two or more of the plurality of electrodes can excite an electric field, detect within the electric field, or excite and detect within the electric field, wherein the device is configured to obtain gastric/reflux bolus data at or near the gastroesophageal junction.

### PRIORITY

The present application is related to, and claims the priority benefit of, U.S. Provisional Patent Application Ser. No. 61/898,596, filed Nov. 1, 2013, the contents of which are hereby incorporated by reference in their entirety into the present disclosure.

### BACKGROUND

The gastrointestinal tract serves to transport nutrients and fluids from the mouth to the site where they are being absorbed in the intestines. The food is swallowed into the esophagus and transported to the stomach for further breakdown before entering the small intestine.

Various diseases of the esophagus disturb or inhibit the mechanical (transport) action and may give rise to symptoms. With malfunction of the esophagus, patients may experience symptoms such as heartburn and pain. Several diseases, such as systemic sclerosis and achalasia, may affect the function of the esophagus, but the so-called “functional diseases” are of particular interest; i.e., functional implies that the cause of the disease is unknown. Hence, a diagnosis cannot be obtained by conventional diagnostic tools such as pressure recordings and medical imaging technologies.

Functional visceral diseases are very common, such as: i) functional (non-cardiac) chest pain with 180,000 new incidences yearly in the US, and ii) gastro-esophageal reflux disease (GERD) affecting 15-20% of the population, and less than a third of all cases can be diagnosed using endoscopy.

In the United States, recent independent surveys indicate that 40% of adults suffer from a heartburn event at least once a month. Out of those approximately 80,000,000 adults in the United States alone, approximately 7% (14,000,000) suffer heartburn events daily. These sufferers are classified as having GERD. Incompetence of the lower esophageal sphincter and decreased motility in the esophagus are known causes of GERD. GERD is generally not seen as a life threatening disorder although about 1% of sufferers go on to develop a condition known as Barrett's esophagus, which is a prestage for esophageal cancer, the fastest growing cancer in the Western World.

Most GERD sufferers seek medical attention, and the treatment of choice is drug therapy. There is a range of prescription and non-prescription drugs that can help. Most of the medications works by reducing or neutralizing the acid produced in the stomach. Although this medication is generally expensive, it is quite effective, with approximately 13% of adults taking indigestion aids twice a week.

Hence, gastrointestinal disorders are perhaps some of the most widespread of all medical ailments and represent a large number of physician office visits each year. Prescription drugs for GERD treatment represent the largest usage sector in the market. This suggests that, at a minimum, the GERD drug treatment market in the U.S. is worth approximately $5.1 billion per year. There is a huge interest in development of new diagnostic technologies due to the high prevalence of these diseases since they are difficult to diagnose with existing technology.

The 25% of patients who do not effectively respond to drug therapy for GERD, as well as patients with chest pain, will usually have tests to evaluate the ability of the esophagus and intestines to clear it food contents. These tests are usually performed in a gastrointestinal motility laboratory by a specialized physiology technician. Most major hospitals with a gastroenterology department would have a motility laboratory, such as university teaching hospitals and tertiary referral centers. The most widely used tests of the esophagus are manometry, pH-recordings, endoscopy, and the proton pump inhibitor (PPI) test. Such tests (especially manometry) have gone through a huge development during recent years with much better axial resolution and presentation of data as color contour plots.

Within the past fifteen years, a new impedance technology (intraluminal impedance) became common to use in gastrointestinal (GI) physiology labs. With this technology, electrical impedance is measured using a number of electrodes on a catheter placed in the esophagus. The electrical impedance changes as a fluid bolus passes by the electrodes, either initiated by a swallow or by reflux of acidic contents from the stomach. Hereby it is possible to trace the bolus and for example determine the velocity thereof. However, the impedance signals have until now been used mainly in a qualitative way, merely showing the tracings of impedance which by themselves are not physiologically useful.

One reason for this lack of methodological development is likely due to lack of research into better use of the signals for physiologically relevant analysis. Another reason is that the electrode spacings, current, and frequencies in the commercially-available systems may not be optimal for more advanced analysis.

In view of the same, devices, systems, and methods for obtaining reflux data/information and/or obtaining other esophageal or gastrointestinal data/information, and using the same to diagnose patient conditions, would be welcome in the marketplace.

### BRIEF SUMMARY

In an exemplary embodiment of a device of the present disclosure, the device comprises an elongated body having a length sufficient so that a distal end of the elongated body can extend to a person's (such as a patient's) gastroesophageal junction while a proximal end of the device, or a connector coupled thereto, is present at the person's mouth, in the person's throat, or outside of the person's mouth and body, and a plurality of electrodes positioned at known distances from one another along the elongated body, wherein some of the plurality of electrodes can excite an electric field, detect within the electric field, or excite and detect within the electric field. In another embodiment, the plurality of electrodes comprises ten or more electrodes. In yet another embodiment, the plurality of electrodes comprises thirty or more electrodes.

In an exemplary embodiment of a device of the present disclosure, the plurality of electrodes are spaced equidistant from one another. In an additional embodiment, the plurality of electrodes are spaced a known distance from one another.

In an exemplary embodiment of a system of the present disclosure, the system comprises an exemplary device of the present disclosure, and a data acquisition and processing system operably coupled to the exemplary device and configured to obtain and process conductance data from the exemplary device. In another embodiment, the system further comprises a connector coupled to the exemplary device and to the data acquisition and processing system. In yet another embodiment, the system further comprises a display operably coupled to one or more of the data acquisition and processing system, the exemplary device, and/or the connector, the display configured to visually depict conductance data and/or data calculated based upon the conductance data.

In an exemplary embodiment of a method of the present disclosure, the method comprises the steps of inserting at least part of an exemplary device of the present disclosure into a patient's esophageal lumen, and operating the device to obtain conductance data within the esophageal lumen. In another embodiment, the operating step comprises a first operating step, the first operating step performed in connection with one or more saline swallows. In yet another embodiment, the first operating step is performed to obtain conductance data to determine at least one parameter of patient's esophagus, such as a local cross-sectional area at or near one or more detection electrodes of the device. In an additional embodiment, the first operating step is performed to determine the presence of a swallowed bolus within the esophageal lumen.

In an exemplary embodiment of a method of the present disclosure, the first operating step is performed to determine the velocity of the swallowed bolus within the esophageal lumen. In an additional embodiment, the first operating step is performed to determine the duration of the presence of the swallowed bolus within the esophageal lumen. In yet an additional embodiment, the first operating step is performed to determine absolute sizing of an esophageal lumen at various locations during swallow. In another embodiment, the first operating step is performed to determine absolute sizing of esophageal wall thickness.

In an exemplary embodiment of a method of the present disclosure, the operating step comprises a second operating step, the second operating step performed to obtain gastric/reflux bolus data. In another embodiment, the second operating step is performed to determine the presence of the gastric/reflux bolus within the esophageal lumen. In yet another embodiment, the second operating step is performed to determine the volume of the gastric/reflux bolus within the esophageal lumen. In an additional embodiment, the second operating step is performed to determine one or more cross-sectional areas of the esophageal lumen generally in the presence of the gastric/reflux bolus.

In an exemplary embodiment of a method of the present disclosure, the second operating step is performed to determine the duration of the presence of the gastric/reflux bolus within the esophageal lumen. In an additional embodiment, the method further comprises the step of diagnosing a condition based upon performing the first operating step and/or the second operating step. In yet an additional embodiment, the condition is selected from the group consisting of gastro-esophageal reflux disease (GERD), acid reflux, a condition relating to improper swallowing, a dilated portion of the esophagus, and an esophageal blockage.

In an exemplary embodiment of a device of the present disclosure, the device comprises an elongated body having a sufficient length so that a distal end of the elongated body can extend to a gastroesophageal junction of a person while a proximal end of the device, or a connector coupled thereto, is present at or distal to a throat of the person relative to the gastroesophageal junction, and a plurality of least ten electrodes positioned at known distances from one another along the elongated body, wherein two or more of the plurality of electrodes can excite an electric field, detect within the electric field, or excite and detect within the electric field, wherein the device is configured to obtain gastric/reflux bolus data at or near the gastroesophageal junction.

In another embodiment, the elongated body has a sufficient length so that the proximal end of the device, or the connector coupled thereto, is present at or distal to a mouth of the person. In yet another embodiment, the elongated body has a sufficient length so that the proximal end of the device, or the connector coupled thereto, is present outside of a mouth and body of the person. In an additional embodiment, the plurality of at least ten electrodes comprises at least thirty electrodes.

In an exemplary embodiment of a device of the present disclosure, each electrode of the plurality of at least ten electrodes is spaced equidistant from one another. In an additional embodiment, the device further comprises one or more sensors positioned along the elongated body, at least one of the one or more sensors configured to obtain data selected from the group consisting of pH data and other chemical-related data. In yet an additional embodiment, at least one electrode of the plurality of at least ten electrodes is configured to obtain pH data and/or other chemical-related data.

In an exemplary embodiment of a system of the present disclosure, the system comprises a device comprising an elongated body having a sufficient length so that a distal end of the elongated body can extend to a gastroesophageal junction of a person while a proximal end of the device, or a connector coupled thereto, is present at or distal to a throat of the person relative to the gastroesophageal junction, and a plurality of least ten electrodes positioned at known distances from one another along the elongated body, wherein two or more of the plurality of electrodes can excite an electric field, detect within the electric field, or excite and detect within the electric field, wherein the device is configured to obtain gastric/reflux bolus data at or near the gastroesophageal junction, and a data acquisition and processing system operably coupled to the exemplary device and configured to obtain and process one or more of conductance data, gastric/reflux bolus data, and/or other data from the device. In another embodiment, the system further comprises a connector coupled to the device and to the data acquisition and processing system, and a display operably coupled to one or more of the data acquisition and processing system, the exemplary device, and/or the connector, the display configured to visually depict the conductance data and/or data calculated based upon the conductance data.

In an exemplary embodiment of a method of the present disclosure, the method comprises the steps of inserting at least part of an device of the present disclosure into a person's esophageal lumen, the device comprising an elongated body having a sufficient length so that a distal end of the elongated body can extend to a gastroesophageal junction of the person while a proximal end of the device, or a connector coupled thereto, is present at or distal to a throat of the person relative to the gastroesophageal junction; and a plurality of least ten electrodes positioned at known distances from one another along the elongated body, wherein two or more of the plurality of electrodes can excite an electric field, detect within the electric field, or excite and detect within the electric field, wherein the device is configured to obtain gastric/reflux bolus data at or near the gastroesophageal junction, and operating the device to obtain conductance data within the esophageal lumen. In another embodiment, the operating step comprises a first operating step, the first operating step performed in connection with one or more saline swallows. In yet another embodiment, the first operating step is performed to obtain conductance data to determine at least one parameter of an esophagus of the person, such as a local cross-sectional area at or near one or more of the plurality of at least ten electrodes of the device. In an additional embodiment, the first operating step is performed to determine the presence of a swallowed bolus within the esophageal lumen. In yet an additional embodiment, the first operating step is performed to determine a velocity of a swallowed bolus within the esophageal lumen.

In an exemplary embodiment of a method of the present disclosure, the first operating step is performed to determine a duration of the presence of a swallowed bolus within the esophageal lumen. In an additional embodiment, the first operating step is performed to determine sizing information selected from the group consisting of sizing of the esophageal lumen at various locations during swallow and absolute sizing of esophageal wall thickness. In yet an additional embodiment, the operating step comprises a second operating step, the second operating step performed to obtain gastric/reflux bolus data. In an additional embodiment, the second operating step is performed to determine a presence and/or a volume of the gastric/reflux bolus within the esophageal lumen. In another embodiment, the method further comprises the step of diagnosing a condition based upon performing the first operating step and/or a second operating step performed after the first operating step. In yet another embodiment, the condition is selected from the group consisting of gastro-esophageal reflux disease (GERD), acid reflux, a condition relating to improper swallowing, a dilated portion of the esophagus, and an esophageal blockage.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a view of a portion of an exemplary device positioned within an esophageal lumen in connection with a swallowed bolus, according to an exemplary embodiment of the present disclosure;

FIG. 2 shows a view of a portion of an exemplary device positioned within an esophageal lumen in connection with a reflux bolus, according to an exemplary embodiment of the present disclosure;

FIG. 3 shows a block diagram of steps of a method, according to an exemplary embodiment of the present disclosure;

FIGS. 4A and 4B show block diagrams of components of a system, according to an exemplary embodiment of the present disclosure;

FIG. 5 shows graphical and tabular data relating to swallow volume and esophageal lumen volume, according to an exemplary embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

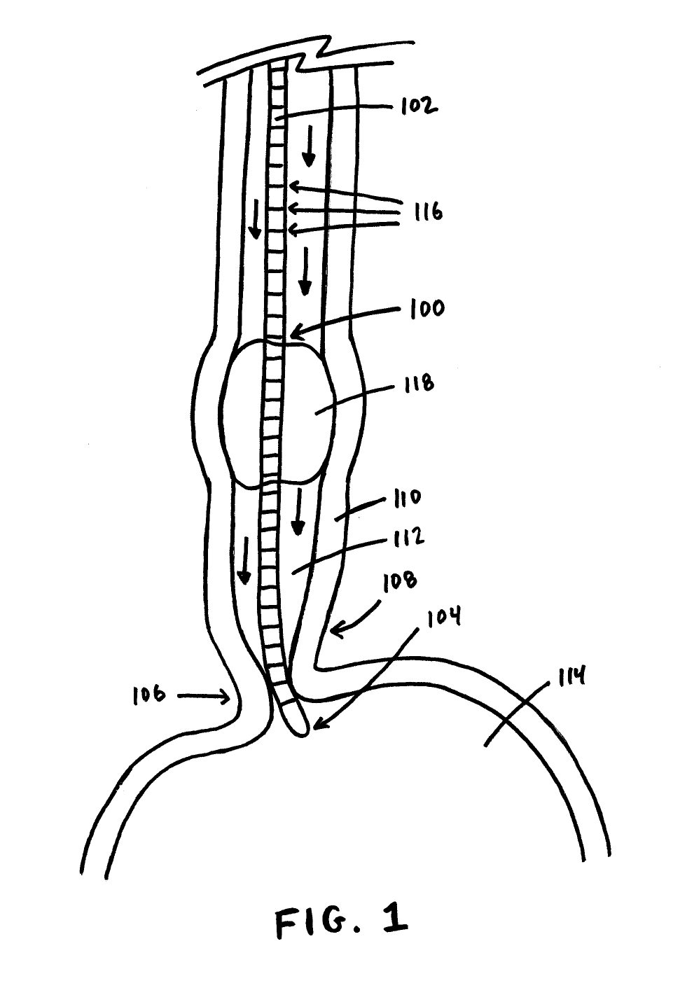

An exemplary device for obtaining volume reflux data/information and/or obtaining other esophageal or gastrointestinal data/information of the present disclosure is shown in FIG. 1. As shown in FIG. 1, device 100 comprises an elongated body 102 having a length sufficient so that a distal end 104 of device 100 can extend to the gastroesophageal junction 106 at the distal end 108 of the esophagus 110 while a proximal end of device 100 (not shown in FIG. 1) or a connector (also not shown) coupled to device 100 is present at a person's mouth, in the person's throat, our outside of the person's mouth and body. The arrows pointing downward within the esophageal lumen 112 indicate the direction of a normal swallowed bolus (food and/or water) flow from the person's mouth (not shown) and into the person's stomach 114 during swallow. FIG. 1 shows a portion of the esophagus 110 and the upper portion of the stomach 114.

An exemplary device 100, as shown in FIG. 1, comprises a plurality of electrodes 116 spanning the majority or all of a length of esophagus 110. In an exemplary embodiment, device 100 comprises at least thirty electrodes 116 positioned along device 100, with said electrodes 116 positioned at or near 1.0 cm from one another. In another embodiment, device 100 comprises between 30 and 40 electrodes 116. In various other embodiments, device 100 comprises more than 10 electrodes 116. Electrodes 116 may be spaced equidistant from one another, such as 0.10″, 0.20″, 0.30″, 0.40″, 0.50″, 0.25 cm, 0.50 cm, 0.75 cm, 1.0 cm, 1.25 cm, 1.50 cm, and/or other distances from one another. Electrodes 116 may also have varied spacings, where certain electrodes 116 are positioned a first distance from one another while other electrodes 116 are positioned a second distance from one another. Electrodes 116, as provided in further detail herein, can be used to obtain conductance data using impedance measurements.

FIG. 1 also shows a swallowed bolus 118 in the esophageal lumen 112, moving downward as indicated by the arrows therein. A localized distension of esophagus 110 is also shown therein, as during swallow of a fluid (a liquid swallowed bolus 118, for example), esophagus 110 locally distends at a location of said swallowed bolus 118 as swallowed bolus 118 moves toward the stomach 114. Also as shown in FIG. 1, esophageal junction 106 is relatively closed around device 100, so to prevent contents of stomach 114 from entering the esophagus 110.

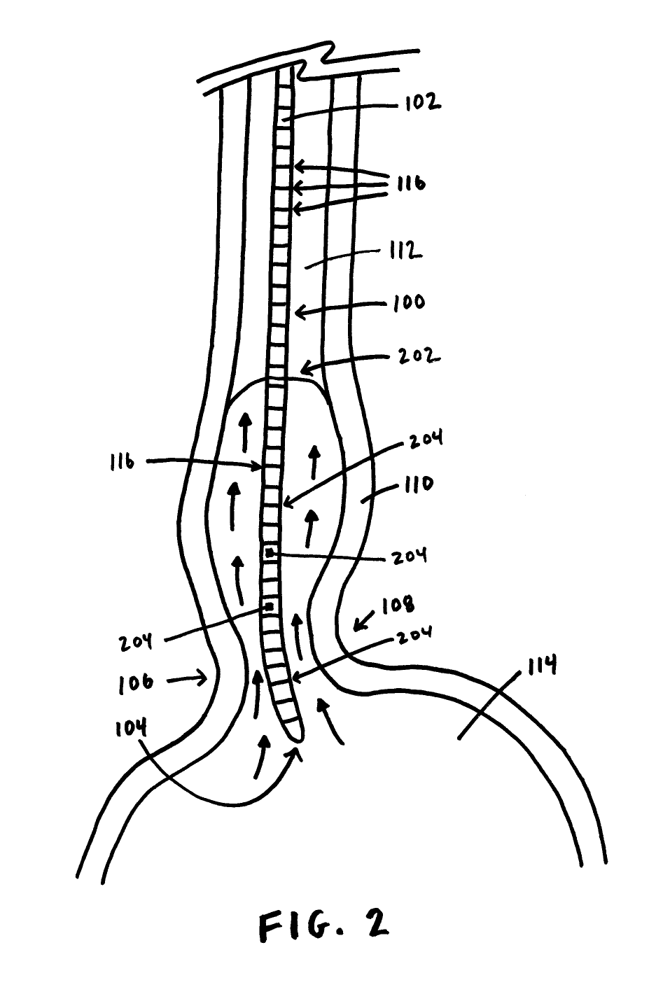

FIG. 2 shows an exemplary device 100 of the present disclosure positioned within an esophageal lumen 112, with a distal end 104 of device extending to the gastroesophageal junction 106. Due to relaxation of the lower esophageal sphincter, for example, gastroesophageal junction 106 can open, as shown in FIG. 2, allowing a gastric bolus 200 (namely contents of the stomach 114) to extend upward (in the direction of the arrows shown within the esophageal lumen 112). Said gastric bolus 200 (also referred to as a reflux bolus), can extend into the esophageal lumen 112, with the relative severity of the reflux being tied to the distance a distal end 202 of gastric bolus 200 can travel within the esophageal lumen 112.

Various devices 100 of the present disclosure can be used to determine reflux severity, volume reflux, and/or obtain other data relating to the esophagus and/or other portions of the gastrointestinal system as described in further detail herein.

In addition, and as shown in the exemplary device 100 of the present disclosure shown in FIG. 2, exemplary devices 100 may include one or more sensors 204 useful to obtain pH data and/or other chemical-related data in connection with gastric bolus 200. The exemplary device 100 shown in FIG. 2 shows various electrodes 116 and sensors 204, noting that in individual device 100 embodiments, devices 100 may a) have a plurality of electrodes 116 and no sensors 204, or b) have a plurality of electrodes 116 and one or more sensors 204. In alternative a), for example, electrodes 116 may be useful to obtain conductance data and not pH data or other chemical-related data, or electrodes 116 may be useful to obtain conductance data and one or both of pH data and chemical-related data. Alternatives a) and b) noted above are not intended to be an exhaustive list of alternatives, as other alternatives consistent with the present disclosure may be contemplated. Furthermore, and as shown in FIG. 2, sensors 204 may be spaced consistent with other electrode 116 spacing (so that electrodes 116 and sensors 204 are consistently or otherwise spaced as referenced herein), and/or sensors 204 may be positioned in a spacing inconsistent with electrode 116 spacing, such as between two electrodes 116, for example.

With respect to using devices 100, and the various electrodes 116 of said devices, conductance data is obtained during operation of said devices 100. The governing relation between the measured total conductance (G_{T}) and cross-sectional area (CSA) at a particular location within a luminal organ is given by the following:

$${G}_{T} = \frac{CSA \cdot α}{L} + {G}_{p} \qquad [ 1 ]$$

where L is a constant determined by the distance between two electrodes 116 used for detection (collecting conductance data), a is the specific electrical conductivity of the local fluid (such as blood), and G is the parallel conductance. In the case of an esophagus 110, and when the esophagus 110 is generally closed (wherein the esophagus 110 is a collapsed tube (i.e., the lumen CSA equals zero), at resting/collapsed conditions,

G_{T}=Gₚ i.e., CSA=0 [2]

As such, the parallel conductance (Gₚ) can be determined in the absence of a swallowed bolus 118 in a resting person/patient. During a swallow, the person/patient can drink a saline fluid/solution (or a fluid having a saline component) in order to determine the luminal CSA in connection with the saline swallowed bolus 118, as follows:

$$CSA = ( {G}_{T} - {G}_{p} ) \frac{L}{{α}_{saline}} \qquad [ 3 ]$$

wherein αₛₐₗᵢₙₑ is the specific conductivity of saline.

Hence, the CSA of the esophagus (esophageal lumen 112) at the various serially placed detection electrodes 116 can be measured to provide not only the absolute dimensions of the esophageal lumen 112, but also the dynamic movement of the swallowed bolus 118 along the esophagus 110. In view of the same, swallowed bolus 118 velocity can be considered as the time of transit of swallowed bolus 118 between two detection electrodes 116 divided by the length traveled.

With respect to reflux, i.e. when substances moves from stomach 114 to esophagus 110, such as in the case of a reflux bolus 200 as shown in FIG. 2, a pair of detection electrodes 116 (having a known spacing L, such as 1.0 mm, for example), to measure α_{reflux}, which is the specific conductivity of the reflux bolus 200. Excitation of certain electrodes 116 and detection of certain electrodes 116 (the same or different from those used to excite a field to obtain the conductance measurements) with such a short spacing (L) will provide the conductivity of the refluxate (reflux bolus 200). Said measurements can be made continuously over time so to provide temporal data with respect to reflux bolus 200 in connection with its severity and duration.

The velocity of reflux is determined as the change in time between two sets of detection electrodes (electrodes 116) and the length between such as:

$${U}_{reflux} - velocity = \frac{Δ T}{L} \qquad [ 4 ]$$

wherein U_{reflux} is the mean velocity of the reflux bolus 200, ΔT is the change in time, and L is the distance between the two pairs of detection electrodes 116, so that one pair of electrodes 116 can sense at a first time and the second pair 116 can sense at the second time, with the temporal difference between the two providing velocity data. The cross-sectional are (CSA) of the esophagus 110 during reflux is determined as given above, namely:

$$CSA = ( {G}_{T} - {G}_{p} ) \frac{L}{{α}_{reflux}} \qquad [ 5 ]$$

wherein G_{T} is the measured total conductance, Gₚ is the parallel conductance, L is the distance between the two pairs of detection electrodes, and α_{reflux} is the specific conductivity of the reflux bolus 200, which is measured continuously. The volumetric flow of reflux (Q) is given by conservation of mass, namely the product of CSA and mean velocity (U):

Q=CSA·U [6]

The volume of reflux (V reflux volume) can then be determined as the integral of volumetric flow rate over the time interval of interest:

V_{reflux volume}=∫₀^{T}Qdt=∫₀^{T}CSA·Udt [7]

The above formulation provides a comprehensive representation of reflux content (conductivity), amount (volume), extent of travel along the esophagus (given the detection electrodes along the length of the esophagus), and speed of reflux (velocity and flow rate).

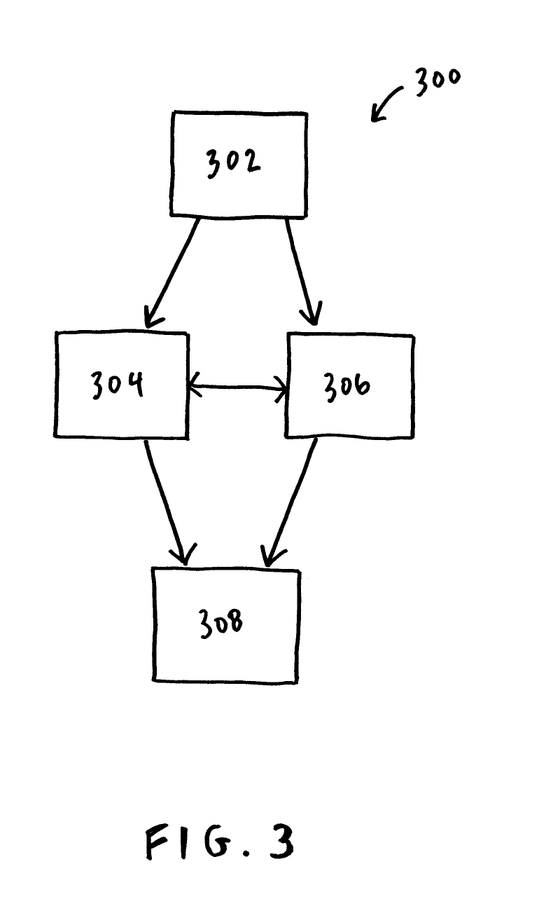

In view of the foregoing, the present disclosure includes disclosure of various devices 100 and methods 300 of obtaining reflux data/information and/or obtaining other esophageal or gastrointestinal data/information. For example, and as shown in the block step diagram of FIG. 3, an exemplary method 300 of the present disclosure includes the step of inserting at least part of an exemplary device 100 of the present disclosure into the esophageal lumen 112 of a patient (an exemplary insertion step 302), and operating said device 100 in connection with one or more saline swallows (an exemplary saline swallow step 304) so (a) to obtain conductance data to determine at least one parameter of the esophagus 110, such as a local cross-sectional area (CSA) at or near one or more detection electrodes 116, (b) to determine the presence of the swallowed bolus 118 within the esophageal lumen 112, (c) to determine the velocity of the swallowed bolus 118 within the esophageal lumen 112, (d) to determine the duration of the presence of the swallowed bolus 118 within the esophageal lumen 112, (e) to determine absolute sizing of an esophageal lumen 112 at various locations during swallow, and/or (f) determine absolute sizing of esophageal wall thickness. Swallow step 304 is optional, but may be performed to obtain data in connection with the swallowed bolus 118 (which can be saline, for example). An exemplary method 300 of the present disclosure may also include the step of operating said device to obtain gastric/reflux bolus 200 data (an exemplary reflux bolus detection step 306), which can indicate (and/or obtain, as applicable) (a) the presence of the gastric/reflux bolus 200 within the esophageal lumen 112, (b) the volume of the gastric/reflux bolus 200 within the esophageal lumen 112 (by calculating various cross-sectional areas within the esophageal lumen 112 in the presence of the gastric/reflux bolus 200 and the overall distance between electrodes 116 used to obtain said CSAs to obtain volume data), (c) one or more CSAs generally in the presence of the gastric/reflux bolus 200, (d) the duration of the presence of the gastric/reflux bolus 200 within the esophageal lumen 112, (e) pH data in connection with the gastric/reflux bolus 200, such as one or more pH measurements of said gastric/reflux bolus 200 at one or more locations along device 100 (using multiple sensors 204, for example, and or electrodes 116 configured to obtain pH data), and/or (f) other chemical-related data in connection with the gastric/reflux bolus 200, such as data to indicate the presence of one or more chemicals within said gastric/reflux bolus 200 at one or more locations along device 100 (using multiple sensors 204, for example, and or electrodes 116 configured to obtain chemical-related data). Step 304, as noted above may also include obtaining pH and/or chemical-related data in connection with a swallowed bolus 118 as well. Reflux bolus detection step 306 is also optional, and as noted in FIG. 3, steps 304 and 306 may be performed separately or in connection with one another, in either order.

Exemplary methods 300 may further comprise the step of diagnosing a condition, which generally includes determining the presence of a condition and/or confirming a suspected and/or prior diagnosis (an exemplary diagnosis step 308) based upon data collected at one or both of steps 304 or 306. Said conditions may include various conditions of the gastrointestinal system, including GERD, acid reflux, conditions relating to improper swallowing, dilated portions of the esophagus, esophageal blockages, and the like. For example, pH data obtained in connection with a gastric bolus 200, such as obtained in reflux bolus detection step 306, may be useful in connection with an exemplary diagnosis step 308, such as identifying one or more conditions based upon an acidic reflux bolus 200 or identifying one or more conditions, such as the presence of basic duodenal contents within reflux bolus 200.

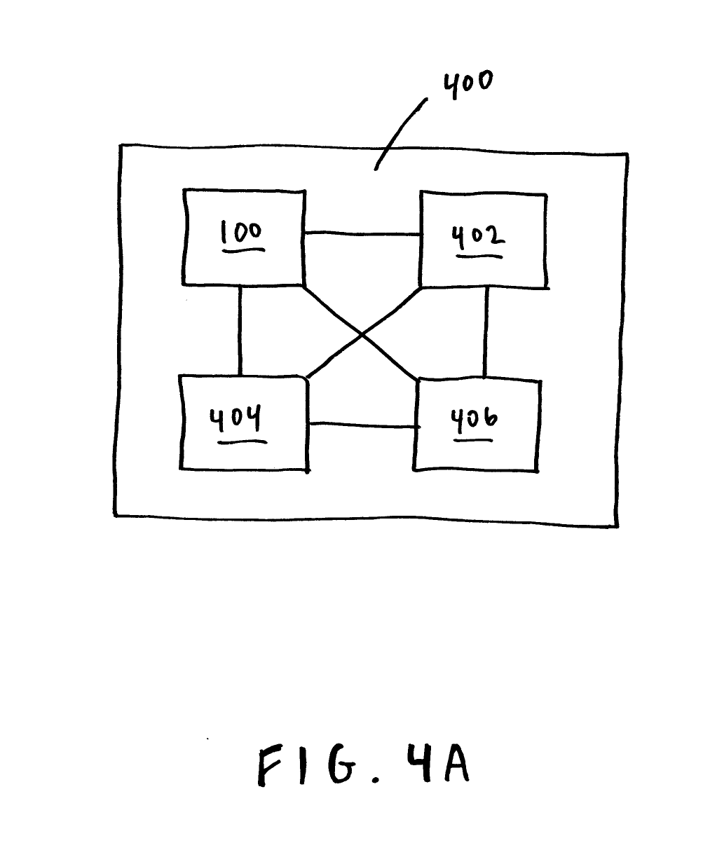

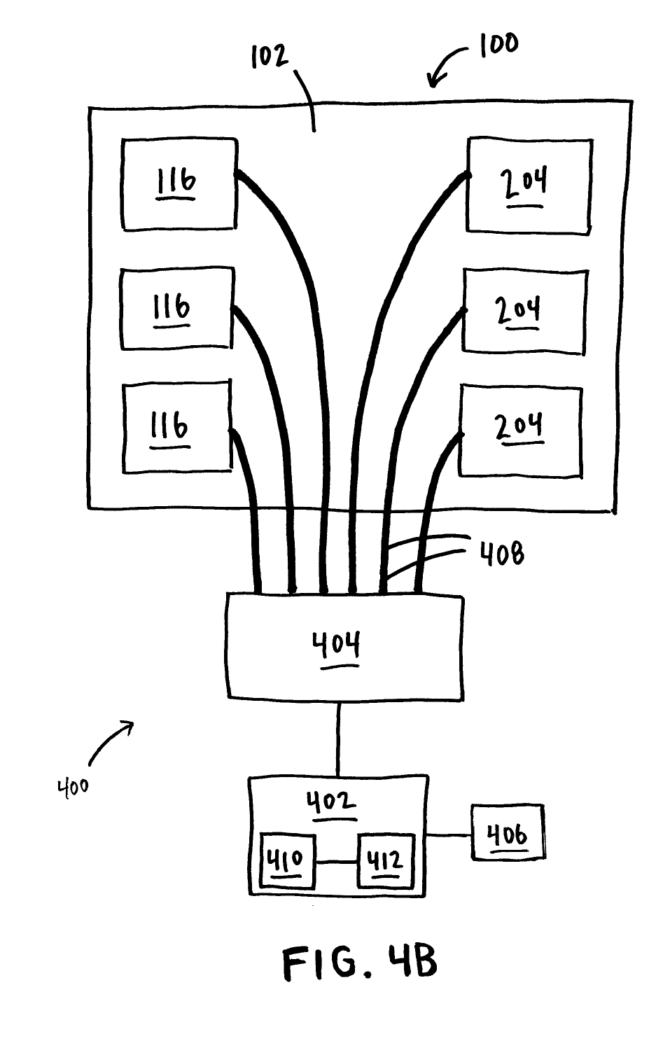

The present disclosure also includes disclosure of various systems 400, as shown in the block diagram of FIG. 4A. As shown therein, an exemplary system of the present disclosure comprises an exemplary device 100 of the present disclosure and a data acquisition and processing system 402 operably coupled to the exemplary device 400, the data acquisition and processing system 402 configured to obtain and process conductance data obtained from the exemplary device 400. Various exemplary systems 400 may also comprise a connector 404 (such as a handle and/or other coupling device) coupled to the exemplary device 100 and/or to the data acquisition and processing system 402. Systems 400 of the present disclosure may also comprise a display 406 operably coupled to one or more of the data acquisition and processing system 402, the exemplary device 100, and/or the connector 404, wherein the display 406 is configured to visually depict conductance data and/or data calculated based upon the conductance data.

FIG. 4B shows certain components of an exemplary device 100 and system 400 of the present disclosure in a block component diagram format. As shown in FIG. 4, an exemplary device 100 may comprise a plurality of electrodes 116 and a plurality of sensors 204. Electrodes 116 and sensors 204, as shown in the figure, have wires 408 coupled thereto so that data from electrodes 116 and sensors 204 may be transmitted therefrom and/or so that operation instructions to electrodes 116 and sensors 204 may be transmitted thereto. Wires 408 are shown as ultimately being coupled to connector 404, noting that wires 408 may also either pass through connector 404 and/or couple directly to data acquisition and processing system 402. Data acquisition and processing system 402, as shown in FIG. 4B, may comprise a processor 410 coupled to a storage medium 412 (such as a hard drive, random access memory, read only memory, a flash drive, etc.) so that instructions can be obtained by or provided to processor 410 from storage medium to operate some or all of device 100.

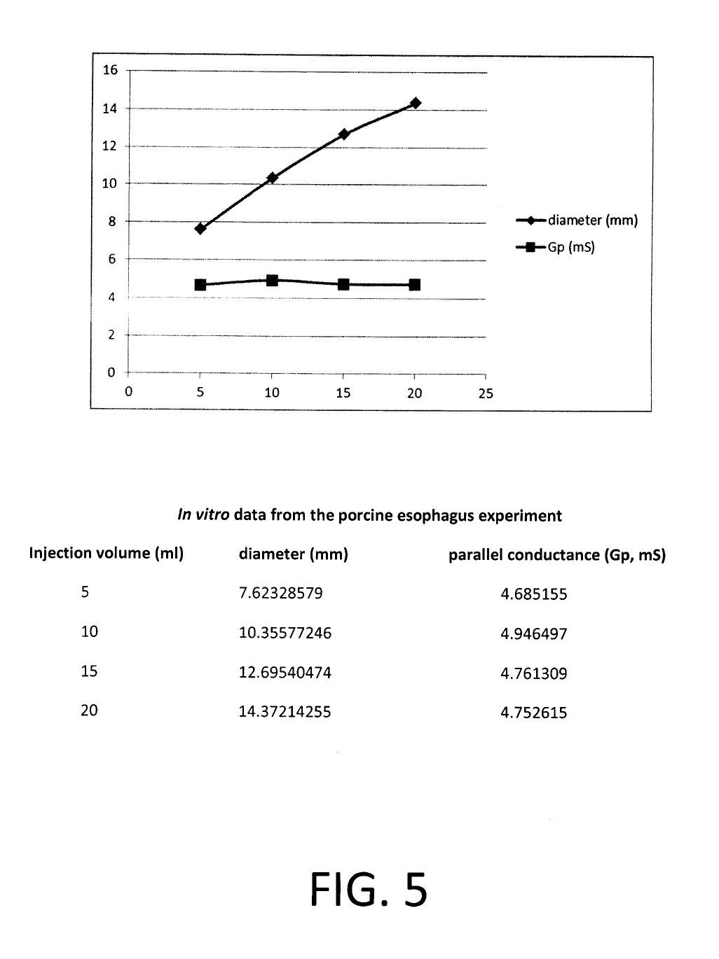

FIG. 5 shows a graph and tabular data obtained using an exemplary device 100 of the present disclosure within a porcine esophagus. As shown therein, FIG. 5 describes the relation between the bolus amount (swallowed bolus 118 volume) and a diameter of the esophageal lumen 112 at the location of detection. The computed diameters, based in part upon the bolus volume, were confirmed as accurate using intravascular ultrasound (IVUS) and show an expected increase in diameter of the esophageal lumen 112 with increased swallowed bolus 118 volume (or volume injection). The data also shows a relatively constant determination of parallel conductance, which is deemed reasonable as generally noted by way of the equations referenced herein.

As referenced herein, it is of special interest to develop a method to determine the various parameters, such as bolus volume, the extent of a bolus moving up the esophagus during reflux episodes, bolus velocity, and/or esophageal wall thickness. The present disclosure provides solutions for generating such parameters from esophageal impedance signals. The analysis will also be useful for studying other parts of the gastrointestinal tract, including the biliary tract system for example, and other hollow organs, such as the urethra and the ureters.

While various embodiments of device and systems for obtaining reflux data/information and/or obtaining other esophageal or gastrointestinal data/information and methods of using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A device, comprising:
an elongated body having a sufficient length so that a distal end of the elongated body can extend to a gastroesophageal junction of a person while a proximal end of the device, or a connector coupled thereto, is present at or distal to a throat of the person relative to the gastroesophageal junction; and
a plurality of least ten electrodes positioned at known distances from one another along the elongated body, wherein two or more of the plurality of electrodes can excite an electric field, detect within the electric field, or excite and detect within the electric field;
wherein the device is configured to obtain gastric/reflux bolus data at or near the gastroesophageal junction.

2. The device of claim 1, wherein the elongated body has a sufficient length so that the proximal end of the device, or the connector coupled thereto, is present at or distal to a mouth of the person.

3. The device of claim 1, wherein the elongated body has a sufficient length so that the proximal end of the device, or the connector coupled thereto, is present outside of a mouth and body of the person.

4. The device of claim 1, wherein the plurality of at least ten electrodes comprises at least thirty electrodes.

5. The device of claim 1, wherein each electrode of the plurality of at least ten electrodes is spaced equidistant from one another.

6. The device of claim 1, further comprising:
one or more sensors positioned along the elongated body, at least one of the one or more sensors configured to obtain data selected from the group consisting of pH data and other chemical-related data.

7. The device of claim 1, wherein at least one electrode of the plurality of at least ten electrodes is configured to obtain pH data and/or other chemical-related data.

8. A system, comprising:
a device, comprising:
an elongated body having a sufficient length so that a distal end of the elongated body can extend to a gastroesophageal junction of a person while a proximal end of the device, or a connector coupled thereto, is present at or distal to a throat of the person relative to the gastroesophageal junction; and
a plurality of least ten electrodes positioned at known distances from one another along the elongated body, wherein two or more of the plurality of electrodes can excite an electric field, detect within the electric field, or excite and detect within the electric field;
wherein the device is configured to obtain gastric/reflux bolus data at or near the gastroesophageal junction; and
a data acquisition and processing system operably coupled to the exemplary device and configured to obtain and process one or more of conductance data, gastric/reflux bolus data, and/or other data from the device.

9. The system of claim 8, further comprising:
a connector coupled to the device and to the data acquisition and processing system; and
a display operably coupled to one or more of the data acquisition and processing system, the exemplary device, and/or the connector, the display configured to visually depict the conductance data and/or data calculated based upon the conductance data.

10. A method, comprising the steps of:
inserting at least part of an device of the present disclosure into a person's esophageal lumen, the device comprising:
an elongated body having a sufficient length so that a distal end of the elongated body can extend to a gastroesophageal junction of the person while a proximal end of the device, or a connector coupled thereto, is present at or distal to a throat of the person relative to the gastroesophageal junction; and
a plurality of least ten electrodes positioned at known distances from one another along the elongated body, wherein two or more of the plurality of electrodes can excite an electric field, detect within the electric field, or excite and detect within the electric field;
wherein the device is configured to obtain gastric/reflux bolus data at or near the gastroesophageal junction; and
operating the device to obtain conductance data within the esophageal lumen.

11. The method of claim 10, wherein the operating step comprises a first operating step, the first operating step performed in connection with one or more saline swallows.

12. The method of claim 11, wherein the first operating step is performed to obtain conductance data to determine at least one parameter of an esophagus of the person, such as a local cross-sectional area at or near one or more of the plurality of at least ten electrodes of the device.

13. The method of claim 11, wherein the first operating step is performed to determine the presence of a swallowed bolus within the esophageal lumen.

14. The method of claim 11, wherein the first operating step is performed to determine a velocity of a swallowed bolus within the esophageal lumen.

15. The method of claim 11, wherein the first operating step is performed to determine a duration of the presence of a swallowed bolus within the esophageal lumen.

16. The method of claim 11, wherein the first operating step is performed to determine sizing information selected from the group consisting of sizing of the esophageal lumen at various locations during swallow and absolute sizing of esophageal wall thickness.

17. The method of claim 11, wherein the operating step comprises a second operating step, the second operating step performed to obtain gastric/reflux bolus data.

18. The method of claim 17, wherein the second operating step is performed to determine a presence and/or a volume of the gastric/reflux bolus within the esophageal lumen.

19. The method of claim 11, further comprising the step of:
diagnosing a condition based upon performing the first operating step and/or a second operating step performed after the first operating step.

20. The method of claim 19, wherein the condition is selected from the group consisting of gastro-esophageal reflux disease (GERD), acid reflux, a condition relating to improper swallowing, a dilated portion of the esophagus, and an esophageal blockage.
